(19) United States
(12) Patent Application Publication
(10) Pub. No.: US 2007/0239002 A1
(43) Pub. Date: Oct. 11, 2007

(54) Superfast, High-Resolution Ultrasonic Imaging Using Coded Excitation

(76) Inventor: Sheikh Kaisar Alam, Somerset, NJ (US)

Correspondence Address: BAKER BOTTS L.L.P., 30 ROCKEFELLER PLAZA, 44TH FLOOR, NEW YORK, NY, 10112-4498, US

(21) Appl. No.: 11/617,679

(22) Filed: Dec. 28, 2006

Related U.S. Application Data
(60) Provisional application No. 60/754,428, filed on Dec. 28, 2005.

(51) Int. Cl. A61B 8/00

(52) U.S. Cl. 600/437

(57) ABSTRACT

An ultrasound imaging scanner has a transducer having a number N of transducer elements. Distinct transducer elements are excited with distinct signals, which can be uniquely coded signal pulses or distinct chirps. The combined echo signal received at a particular transducer is correlated with the excitation signal applied to the particular transducer element to isolate its individual echo.

### CROSS-REFERENCE TO RELATED APPLICATIONS

This application claims the benefit of U.S. provisional patent application Ser. No. 60/754, 428 filed Dec. 28, 2005, which is hereby incorporated by reference herein in its entirety

### FIELD OF THE INVENTION

The present invention generally relates to imaging systems using a vibratory energy beam such as an ultrasonic beam. In particular, this invention relates to a method and apparatus for superfast imaging employing coded excitation and synthetic aperture techniques, for sonography or ultrasound imaging of the human anatomy for medical diagnosis.

### BACKGROUND OF THE INVENTION

In conventional ultrasound imaging, high-frequency sound waves are deployed to image objects (e.g., internal human body organs). High frequency acoustic waves are produced and detected using one or more suitable ultrasonic transducers. An ultrasound imaging system typically uses array transducers, in which array elements are excited in a pre-determined sequence to generate ultrasound beams and create images. (See e.g., FIG. 1). To create one focused ultrasound beam, more than one element is typically excited during transmission as well as during reception. (See e.g., FIG. 2). The transmitted ultrasound beam is focused and steered by serially activating the appropriate elements either in a linear (e.g. in a linear array) or in a sector format (e.g. in a curved array). Focusing on receive is performed by applying appropriate delays to the received echo signals at active elements during receive before summing the signals. However, the scanning process is slow because of the size or number of elements in the transducer array. Current state-of-the-art transducers can have 512 or more elements, which limits the achievable frame rate to typically 40 or fewer frames per second.

Attempts have been made to increase the frame rate of ultrasound scanners. For example, Sandrin et al. have designed an “ultrafast” imaging scanner based on a “synthetic aperture” approach. (See L. Sandrin, S. Catheline, M. Tanter, X. Hennequin, and M. Fink, “Time-resolved pulsed elastography with ultrafast ultrasonic imaging,” Ultrasound Imag., Vol. 21, No. 4, pp. 259-72, 1999). The scanner generates a single, plane ultrasound wave in transmission mode; all or nearly all transducer elements are excited simultaneously to create the plane wave. Thus, the entire field of view is insonified by a single transmitted pulse. Similarly in receive mode, all transducer elements are activate simultaneously. The returned echo signals, received by the transducer elements, are then appropriately phase delayed and summed (post-processing) to synthesize ultrasonic beams and their translation and steering. In this scanner, summation is performed after the echo signals are completely received at all transducer elements. Because all the elements do not need to be excited separately (or in groups) for a single scan plane, the frame rate can be 2 to 3 orders of magnitude faster than is achieved in typical commercial scanners that has to transmit separate focused scan vectors to generate each scan line in an image. However, unlike conventional scanners, the Sandrin method does not focus the transmitted plane wave. Thus, the overall ultrasound beams are wider and has larger side lobes, which degrades lateral resolution and also can introduce artifacts.

O'Donnell describes a coded-excitation scheme to increase the penetration depth of vibratory energy, e.g., in ultrasound imaging. (See Matthew O'Donnell, “Coded excitation for transmission dynamic focusing of vibratory energy beam,” U.S. Pat. No. 5,014,712). In O'Donnell's coded-excitation scheme several coded excitation signal sets, each for a range of depths, are utilized to excite an array of transducer elements. Each different set has signals with a different code, occurs in a different excitation time interval, such that the ultrasound beam is focused into the sample at successively smaller distances along a selected ray path from the array. Thus, during simultaneous transmission, each active element transmits the same code (our invention, as described later, uses a plurality of codes during a given simultaneous transmission). The returned echo signals, which are received by the transducer elements, are processed and all channels are coherently summed for dynamic receive focus. Then, the summed signals are cross-correlated with reference signals, which are the coded signals of the set used for the associated excitation time interval, to recover a response signal substantially only from a volume of the sample at that depth or distance associated with the associated excitation time interval.

This invention improves the imaging speed or frame rate of ultrasound imaging scanners using a coded-excitation scheme in conjunction with synthetic aperture.

### BRIEF DESCRIPTION OF THE DRAWINGS

Further features of the invention, its nature, and various advantages will be more apparent from the following detailed description of the invention and the accompanying drawings, wherein like reference characters represent like elements throughout, and in which:

FIG. 1 is a schematic illustration of an ultrasound transducer arrangement in a conventional ultrasound scanner. In operation, the transducer elements are serially excited with identical transmit pulses.

FIG. 2 is a schematic illustration of a conventional transducer arrangement in a conventional ultrasound scanner. In operation, the transducer elements are simultaneously excited with identical transmit pulse.

FIG. 3 is a schematic illustration of a transducer arrangement in a super fast ultrasound scanner, in accordance with the principles of the present invention. In operation, the transducer elements are simultaneously excited with uniquely coded transmit pulses in the super fast scanner.

FIG. 4 is a schematic illustration of a procedure at transducer element n to extract only the echo that was due to the emitted (uniquely coded) pulse n from itself (element n).

FIG. 5 is a schematic block diagram showing a digital ultrasound imaging system in accordance with the preferred embodiment of the invention. In operation, coded excitation pulses are simultaneously transmitted from transducer elements 300; the returned signals are received simultaneously at elements 300; the received signals are converted to digital form at A/D converter 500 and are stored at random access memory 600; the signal processor 800 (which includes cross-correlator 400 to extract the “self” echo signals) processes the echo signals for display at monitor 900. The entire system is controlled and synchronized by control signal 700, typically in conjunction with a master clock.

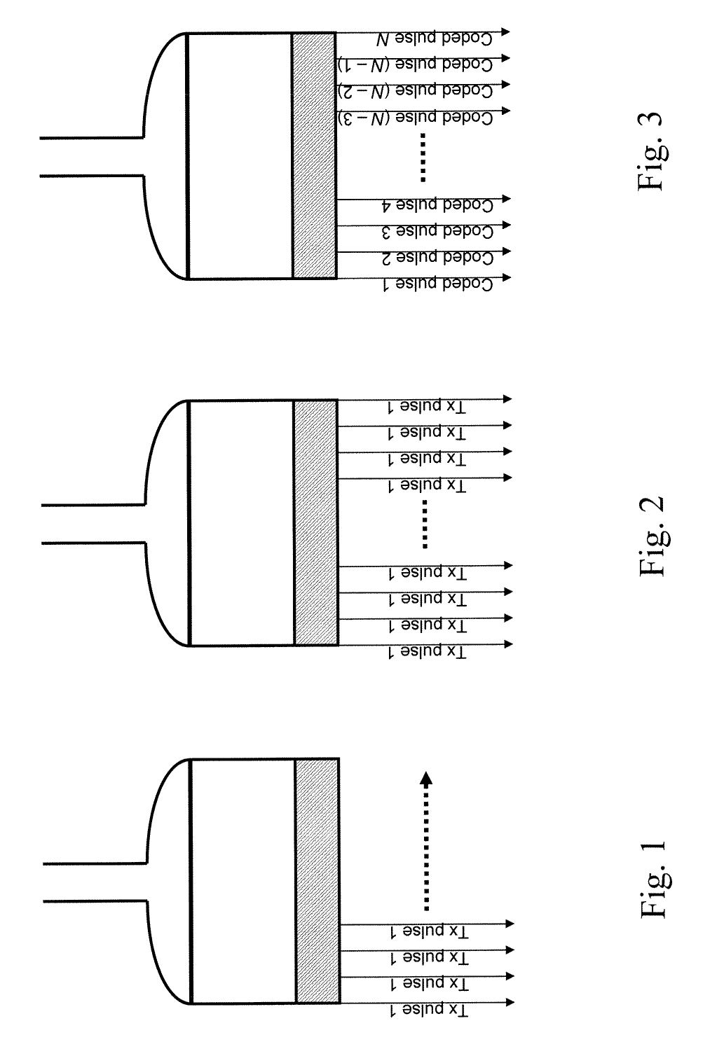

### DESCRIPTION OF THE INVENTION

Ultrasound imaging systems and methods are provided for rapid imaging. The systems and methods can be advantageously used to obtain high frame-rate and accurate views of objects (e.g., internal human body organs). The systems and methods are based on reducing processing/imaging time by use of a plurality of codes (i.e. coded signals) during any simultaneous transmission of signals by transducer elements. This is in contrast to conventional ultrasound imaging systems and methods in which appropriate elements are serially fired to generate ultrasound beams one by one. The inventive systems and methods can achieve very high frame rates.

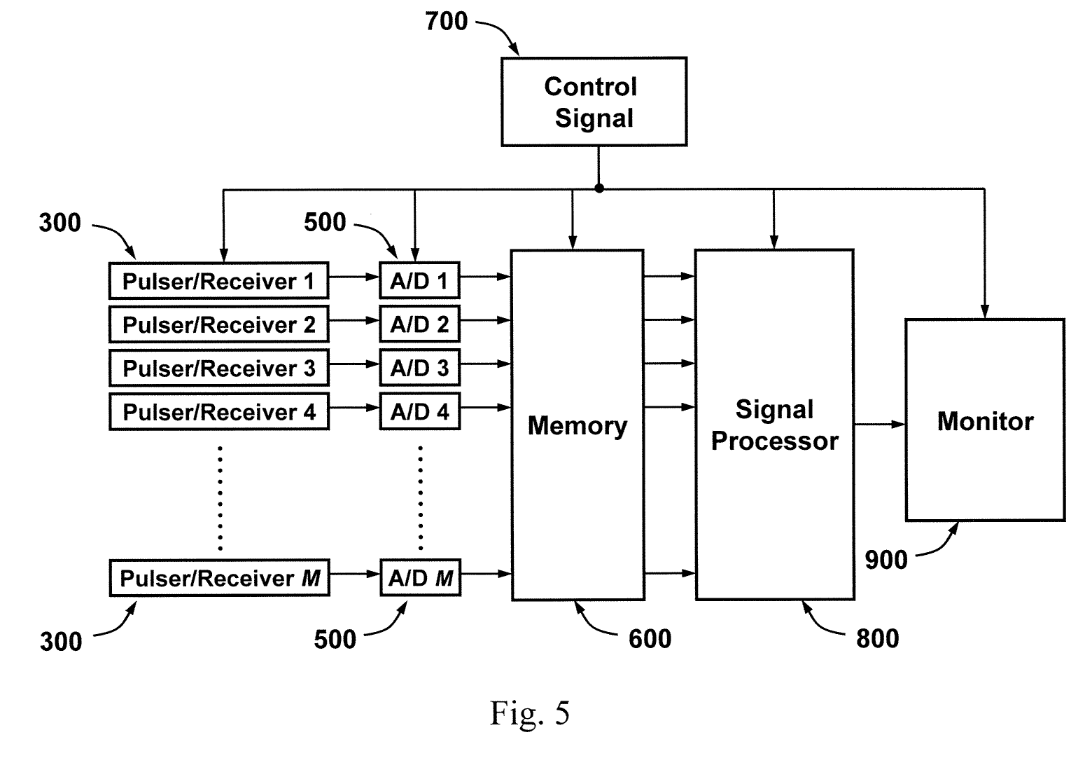

The present invention (systems and methods) exploits the attributes of coded excitations utilized during simultaneous transmissions at all elements (each is a pulser/receiver) of a transducer array 300 (FIGS. 3 and 5). A plurality of unique digital codes (e.g., M different codes) are transmitted via each element, received, and decoded. All transducer array elements are excited simultaneously during transmission. However, unlike the Sandrin method in which all elements emit the same excitation pulse, in the inventive systems and methods each element transmits a unique, long, coded signal ( i.e., a unique signature is encoded on each transmitted signal by repeating a specific pattern of 1's and 0's or 1's and −1's (digital coding)). The unique codes are designed so that the “inter-code” correlations (and nonzero lag “intra-code” correlations) are many orders of magnitude lower than zero lag “intra-code” correlations.

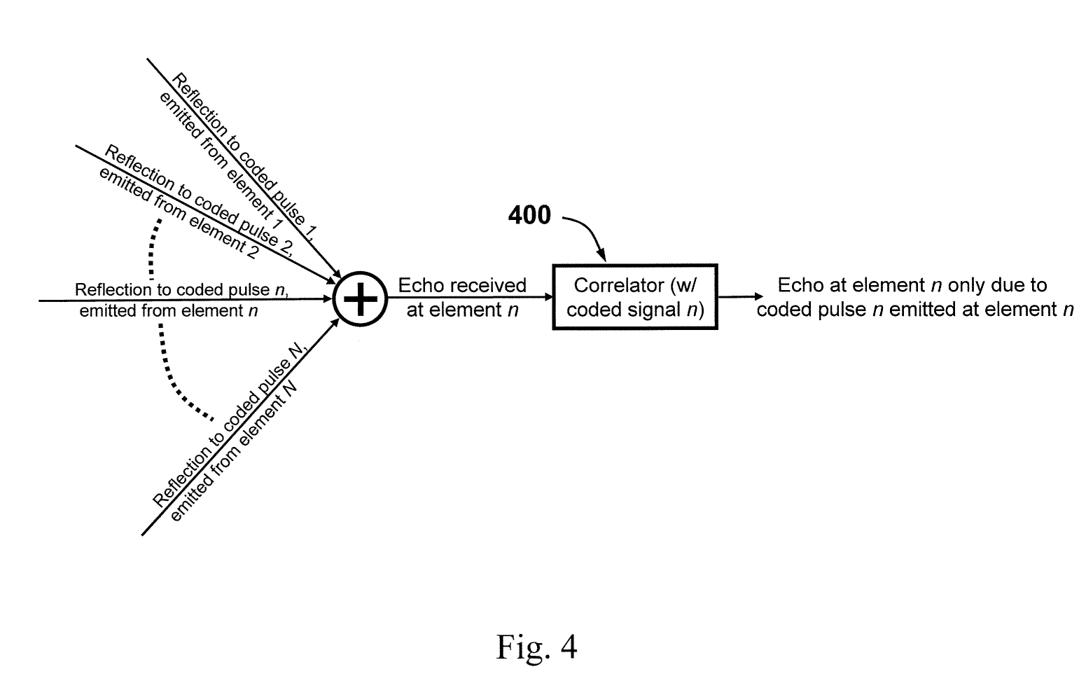

In the receive mode, all transducer array elements 300 receive simultaneously. The “total” echo signal received at each element (e.g. at element eₙ) consists of echoes of signals transmitted by itself and all other elements. (See e.g., FIG. 4). To extract the “self” echo signal, which is due to the signal transmitted by element eₙ itself, from the combined echo signal received at element eₙ, the total signal received at each element is cross-correlated with the uniquely coded signal transmitted at that element (using, for example, a suitable cross-correlator 400, which is a part of signal processor 800 in FIG. 5). Because the inter-code correlation is minimal, the output signal from the cross-correlator (matched-filter) will consist only of the echo resulting from the transmitted signal from an element itself.

According to the present invention, for an N-element transducer, each element will receive echoes from all N elements, creating a set of N² returned echo signals. Each of these of N² returned echo signals can be appropriately phase delayed and summed to focus (during receive) at different points in the scanned plane. For transmit focusing or other beam-controlling purposes, individual transducer elements may be optionally excited with a summation of more than one coded pulse with focusing performed by applying various delays during transmission at individual elements. In other word, more than one element may be optionally excited in transmit or receive mode to synthesize transmit or receive beams.

The invention for fast imaging may be implemented in ultrasound scanners for medical applications. It will be understood, however, that the principles of the invention for fast imaging are equally applicable in any application (medical or otherwise) involving any coherent vibratory energy beam (e.g., RADAR and SONAR). Further, while the invention is described herein the context of linear or curved array configurations of transducer elements, it will be understood that the principles of the invention are applicable to all types of multi-element transducer or antennas, including, for example, annular and multi-element, two-dimensional, phased arrays and antennas. Further, the disclosed methods can be easily applied or extended with appropriate modifications (if necessary) to other ultrasonic imaging situations (e.g., motion/flow imaging, harmonic imaging, etc.).

For two-dimensional array transducers (and capable scanners), the coding method may be used to reconstruct a three-dimensional volumetric image rather than just a two-dimensional plane image (e.g. of a conventional linear array scanner). For a two-dimensional array transducer with M elements by N elements, up to M×N unique codes may be utilized.

In operation, if the required number of unique codes is higher than the number of available codes satisfying use requirements (e.g. the most important being high intra-code and low inter-code correlation), then the transmission may be done in multiple steps or bursts (e.g., in p steps, p >1). For example, if the number of available codes is M and the required number of codes is N (M <N), in each of the p steps or bursts only M elements will be excited or activated in a burst with M coded signals. After all the echoes from the burst are received by those same multiple of elements, the next set of elements will be excited and their echoes received. This process will continue until all the elements have been excited. Thus, a minimum of p bursts are necessary, instead of a single burst, where

$$p = CEIL ( \frac{N}{M} ) ,$$

where CEIL is a function that rounds toward infinity. In such situations, the invention advantageously allows the frame rate to be increased by a factor of M.

In typical implementations, contiguous elements of the transducer array may be excited for each burst. However, patterns of non-contiguous elements of the transducer array may be used in other implementations. For example, firing or activation of alternating patterns of every second, third, or fourth elements may be selected to alter the transmit-receive beam shapes. The excitation of contiguous or non-contiguous elements of the transducer array in bursts may be advantageous in certain applications even when M≧N.

The codes used for the inventive transducer arrangements may include digital codes used in spread spectrum communications with suitable characteristics (e.g., length) for super-fast imaging applications. Such codes include, for example, Barker code, Golay code, M-Sequence codes, etc. However, the Barker code, commonly used for pulse compression of radar signals, has a maximum length of 13, which makes the Barker code potentially unsuitable for super-fast ultrasonic imaging applications. In contrast, Golay codes, which have the advantage of adequate length and zero side lobes, may be unsuitable for super-fast imaging because Golay codes require two transmissions for every ultrasound beam, thereby reducing the achievable frame rates by half. Properly generated, long M-sequences codes are an example of codes that are suitable for super-fast imaging applications under consideration here.

Co-pending and commonly assigned U.S. patent application Ser. No. 11/136,223, entitled “System and Method for Design and Fabrication of a High Frequency Transducer,” describes use of a high-frequency annular transducer in conjunction with synthetic aperture method to increase the depth of penetration, which is very important at high frequencies. For completeness of description, the aforementioned application Ser. No.11/136,223 is hereby incorporated by reference herein in its entirety. In method described in the referenced application, each annular element is individually excited and the return signals are received and recorded at all elements. After all elements have been serially excited, the received signals can be appropriately delayed and summed to synthesize an ultrasonic beam. Although this method permits increasing penetration depth by improving the signal-to-noise ratio (SNR), a total of N transmissions are necessary for an N-annuli array. However, in accordance with the present invention, if all elements are excited simultaneously, each with a unique, uncorrelated code, and all elements receive the combined echo-signals from the targeted tissue, the “self” echo signal, which is due to the signal transmitted by annulus a, itself, can be extracted from the combined echo signal received at annulus an, (e.g., using a suitable cross-correlator 400). Thus, the depth of penetration can be increased without increasing scanning time.

In an alternate embodiment of the inventive systems and methods, unique chirps versus non-axial direction (instead of unique digitally coded excitations versus non-axial direction) may be used, perhaps with some loss of resolution, for simultaneous transmissions by all elements of a transducer array 300 (FIG. 3 and 5). In this embodiment, every transmitted beam is represented by a unique chirp at a different frequency band. For example, for a transducer having a frequency bandwidth (ƒ₁-ƒ_{N}), a first beam can be a chirp signal in the frequency band (ƒ₁-ƒ₂), a second beam can be a chirp signal at the next band (ƒ₂-ƒ₃) . . . and the (N-1)^{th} beam can be a chirp signal in the frequency band (ƒ_{N-1} to ƒ_{N}). The number of unique beams can be doubled by reversing the chirp after the (N-1)^{th} beam (e.g., a N^{th} beam in the frequency band (ƒ₂-ƒ₁), . . . and a 2(N-1)^{th} in the frequency band (ƒ_{N}-ƒ_{N-1})).

Like the case of unique coded excitations (FIG. 4), the received echo signal from the simultaneous transmission of the chirp signals by an ultrasound transducer are processed by a correlator (e.g., correlator 400). The received signal may be separated into separate beams, for example, by correlating with chirps ƒ₁ to ƒ₂ (beam 1), ƒ₂ to ƒ₃ (beam 2) . . . fƒ_{N-1} to ƒ_{N} (beam N−1), ƒ₂ to ƒ₁ (beam N), ƒ₃ to ƒ₂ (beam N+1), . . . , and ƒ_{N} to ƒ_{N-1} (beam 2N−2), respectively.

If rapid frame rates are not essential, image-noise can be reduced in low SNR environments by averaging the signal from each plane from N simultaneous, coded bursts, instead of creating one image by generating N individual, non-simultaneous beams.

While there have been described herein preferred/exemplary embodiments of the present invention, those skilled in the art will recognize that further changes and modifications may be made thereto without departing from the spirit of the invention, and it is intended to claim all such changes and modifications that are within the spirit of the invention.

It also will be understood that the systems and methods of the present invention can be implemented using any suitable combination of hardware and software. The software (i.e., instructions) for implementing and operating the aforementioned systems and methods can be provided on computer-readable media, which can include without limitation, firmware, memory, storage devices, microcontrollers, microprocessors, integrated circuits, ASICS, on-line downloadable media, and other available media.

1. An ultrasound imaging system, comprising:
an array ultrasound transducer comprising a plurality of N transducer (pulser/receiver) elements, for transmitting ultrasonic waves and detecting returned ultrasound echo-signals from ultrasound scatterers in a test material;
means coupled to said array-transducer for pulsing N transducer elements, separately or simultaneously, with distinct, unique, uncorrelated coded signals;
means for receiving signals at N transducer elements in response to ultrasound echoes due to the transmit events;
means for analog-to-digital conversion coupled to said transducer elements to convert each of said received echo-signals to a digital signal;
means for isolating the echo corresponding to signal transmitted by the particular transducer element (“self-echo”) by employing a matched filter that correlates the received signal with the unique code associated with the respective transducer elements;
means for receive-focusing (beam forming) the signals using synthetic aperture employing appropriate delay and summation; and
a monitor for displaying the resultant image of said beam formed signals.

2. The ultrasound imaging system of claim 1, wherein the distinct, unique, uncorrelated coded signals comprises a set of uniquely coded signal pulses.

3. The ultrasound imaging system of claim 1, wherein the distinct, unique, uncorrelated coded signals comprise a set of distinct digitally coded signal pulses.

4. The ultrasound imaging system of claim 1, wherein the distinct, unique, uncorrelated coded signals comprise a set of distinct M-Sequence coded signal pulses.

5. The ultrasound imaging system of claim 1, wherein the transducer has a frequency bandwidth F, and wherein the set of distinct signals comprises a set of distinct chirp signals in bandwidth F.

6. The ultrasound imaging system of claim 1, wherein the means coupled to said array-transducer for pulsing N transducer elements comprises an arrangement for exciting the transducer elements with uniquely coded signal pulses such that intercode or nonzero intra-code signal correlations are at least a magnitude smaller than zero lag intra-code signal correlations.

7. The ultrasound imaging system of claim 1, wherein the means coupled to said array-transducer for pulsing N transducer elements and the means for receiving signals at the N transducer elements comprise an arrangement for exciting a programmable selection of transducer elements in transducer transmit or receive modes to synthesize transmit and receive beams, respectively.

8. The ultrasound imaging system of claim 1, wherein the means for isolating the echo and the means for beam forming comprise a digital signal processor including a signal correlator.

9. The ultrasound imaging system of claim 1, wherein the means coupled to said array-transducer for pulsing N transducer elements comprises an arrangement for applying a sum of at least two distinct signals and/or phase delay to a transducer element.

10. The ultrasound imaging system of claim 1, wherein the means for receiving signals at N transducer elements comprises an arrangement for applying phase delay to received echo signals and summing the received echo signals to focus the received signal from different points in a scanned plane.

11. The ultrasound imaging system of claim 1, wherein the distinct, unique, uncorrelated coded signals comprise a set of M distinct signal pulses with M less than N, and wherein the means coupled to said array-transducer for pulsing N transducer elements comprises an arrangement for exciting the transducer elements in a pattern of bursts of M or less transducer elements at a time.

12. A method for ultrasound imaging, comprising:
deploying an array ultrasound transducer comprising a plurality of N transducer (pulser/receiver) elements, for transmitting ultrasonic waves and detecting returned ultrasound echo-signals from ultrasound scatterers in a test material;
pulsing the N transducer elements, separately or simultaneously, with distinct, unique, uncorrelated coded signals;
receiving signals at the N transducer elements in response to ultrasound echoes due to the transmit events;
converting each of said received echo-signals to a digital signal;
isolating the echo corresponding to signal transmitted by the particular transducer element (“self-echo”) by employing a matched filter that correlates the received signal with the unique code associated with the respective transducer elements;
receive-focusing (beam forming) the signals using synthetic aperture employing appropriate delay and summation; and
displaying the resultant image of said beam formed signals.

13. The method claim 12, wherein the distinct, unique, uncorrelated coded signals comprise a set of uniquely coded signal pulses.

14. The method of claim 12, wherein the distinct, unique, uncorrelated coded signals comprise a set of distinct digitally coded signal pulses.

15. The method of claim 12, wherein the distinct, unique, uncorrelated coded signals comprise a set of distinct M-Sequence coded signal pulses.

16. The method of claim 12, wherein the transducer has a frequency bandwidth F, and wherein the distinct, unique, uncorrelated coded signals comprise a set of distinct chirp signals in bandwidth F.

17. The method of claim 12, wherein the distinct, unique, uncorrelated coded signals comprise uniquely coded signal pulses such that intercede signal correlations are at least a magnitude smaller than zero lag intra-code signal correlations.

18. The method of claim 12, wherein pulsing the N transducer elements and receiving signals at the N transducer elements comprise exciting a selection of transducer elements in transducer transmit or receive modes to synthesize transmit and receive beams, respectively.

19. The method of claim 12, wherein isolating the echo corresponding to signal transmitted by a particular transducer element comprises correlating a signal received at the particular transducer element with the signal used to pulse the particular transducer element.

20. The method of claim 17, further comprising applying a sum of at least two distinct signals and/or phase delay to a transducer element.

21. The method of claim 17 wherein receiving signals at N transducer elements comprises applying phase delay to received echo signals and summing the received echo signals to focus the received signal from different points in a scanned plane.

22. The method of claim 1, wherein the distinct, unique, uncorrelated coded signals comprise a set of M distinct signal pulses with M less than N, and wherein the pulsing the means pulsing the N transducer elements comprises exciting the transducer elements in a pattern of bursts of M or less transducer elements at a time.

23. Computer readable media comprising instructions to perform the steps of the method of claim 1.
